(19) United States
(12) Patent Application Publication
(10) Pub. No.: US 2012/0214222 A1
(43) Pub. Date: Aug. 23, 2012

(54) METHOD FOR MANUFACTURING A DEVICE FOR REGENERATING BIOLOGICAL TISSUES, PARTICULARLY FOR REGENERATING TISSUES OF THE CENTRAL NERVOUS SYSTEM

(76) Inventors: Alessandro Sannino, Lecce (IT); Pietro Mortini, Segrate (IT)

(21) Appl. No.: 13/503,208

(22) PCT Filed: Oct. 7, 2010
(86) PCT No.: PCT/EP2010/065048

§ 371 Date: Apr. 20, 2012

(30) Foreign Application Priority Data
Oct. 20, 2009 (IT) M12009A001804

(51) Int. Cl. C12M 1/00; B29C 35/16

(52) U.S. Cl. 435/283.1; 264/28

(57) ABSTRACT

A method for manufacturing a device for regenerating biological tissues, particularly for regenerating tissues of the central nervous system, and a device that can be manufactured with said method, the device comprising an outer sheath based on collagen which is substantially tubular and can be interposed between the endings of a biological tissue to be regenerated, and at least one supporting element based on collagen, which is accommodated inside the outer sheath.

### TECHNICAL FIELD

The present invention relates to a method for manufacturing a device for regenerating biological tissues, particularly for regenerating tissues of the central nervous system, and to a device that can be manufactured with such method.

### BACKGROUND ART

Currently, in the medical field, regenerative medicine is becoming established as a therapeutic method for treating several types of lesions. In this modern approach, closing of the wound is achieved by means of the synthesis of scar tissue; particularly in induced regeneration, a bioactive structure is arranged in the wound, modifying the original healing and repair mechanism and inducing regeneration of physiological tissue.

The regeneration devices used, known in the technical jargon as “scaffold”, have an essential role in this process because they act both as physical supports and as guides for tissue growth and supply adapted stimuli, acting as insoluble regulators of cellular function.

Scaffolds, with appropriate composition, structural, mechanical and degradation characteristics, allow therefore a regenerative healing process to take place.

In particular, the manufacturing of scaffolds with pores oriented along a specific direction are used in the tublation technique, which consists in the use of a tubular structure to reconnect damaged tissue endings.

The characteristics of the pores of the scaffold, which generally influence the success of the scaffolds as regenerative guides, comprise the void fraction (the percentage of porosity), pore diameter distribution and pore interconnectivity.

For applications related to the peripheral nervous system, it has been observed that the orientation of the pores of the scaffold plays a critical role in its performance. Studies concerning the peripheral nervous system, but also the central nervous system, have demonstrated that structures oriented along an axis have a strong regenerative activity.

In fact, scaffolds made of collagen in which the pores are aligned along an axis are known and are used both for lesions of the peripheral nervous system and for lesions of the central nervous system such as, for example, spinal cord lesions.

In particular, porous scaffolds of the known type are obtained by slow immersion of a suspension of biocompatible material, such as collagen and glycosaminoglycans, in a freezing bath and by subsequent freeze-drying.

It has been verified that the immersion rate and the temperature of the freezing bath affect significantly the dimensions of the pores and their orientation in the matrix.

Experimental tests have shown that for cylindrical scaffolds with a diameter from 1.5 millimeters to 3.8 millimeters, only particular combinations of immersion rate and freezing temperature lead to axially oriented pores.

For example, US Patent application 2008/0102438 discloses a method for manufacturing tubular scaffolds based on collagen that have a gradient of porosity and of pore size that lies in a radial direction, a pore distribution that is oriented radially and an outer surface that is permeable to proteins and impermeable to cells. In the method described in the above-mentioned patent, a suspension of collagen is introduced in a mold until such mold is filled. The mold is spun about its own axis so as to cause the sedimentation of the suspension and create a hollow tubular structure in its interior.

For creating the porosity inside the hollow tubular structure, a portion of the components that constitute the suspension are first immobilized and then removed.

In particular, in the case of a water-based collagen suspension, in order to immobilize and subsequently remove the components, one uses first the immersion in liquid nitrogen and then lyophilization, known in the technical jargon as “freeze-drying”.

With this method, tubular scaffolds of collagen are obtained with a gradient in the porosity and size of the pores in a radial direction, a pore distribution oriented in a radial direction, and an outer surface that is permeable to proteins and not to cells.

This method allows simple and precise control of the geometry and porosity of the tubular structure.

However, scaffolds of the background art, used for example in peripheral nerve regeneration, are constituted by a tubular body with a single direction of orientation of the porosity, which is not sufficient to ensure correct regeneration in the case of lesions that affect, for example, the spinal cord.

In the case of applications concerning the regeneration of the spinal cord, in fact, the scaffold, besides providing a connection between the two severed endings, must be also capable of reproducing the particular architecture of the spinal cord, which is characterized by an outer portion of white matter and two internal lobes of gray matter.

Despite the fact that the presence of a connection between the two severed endings is sufficient to induce regeneration of the damaged tissue, it has been noted that the microstructural, mechanical and composition characteristics of the tubular structure proper and of the material inserted in the cavity of the tubular structure influence significantly the quality of the regeneration.

### DISCLOSURE OF THE INVENTION

The aim of the present invention is to provide a device for regenerating biological tissues and the respective manufacturing method, particularly for regenerating tissues of the central nervous system such as, for example, the spinal cord.

Within this aim, an object of the invention is to provide a regeneration device capable of protecting the site of the implant from the infiltration of external tissue, while remaining permeable to cells from the inside outward, and at the same time having on its inside such a structure as to facilitate axonal regrowth of the right and left lobes of lesioned marrow, by providing the axons with adequate physical and chemotactic support.

Another object of the invention is to provide a regeneration device that is highly reliable, relatively easy to provide and which has competitive costs.

This aim and these and other objects that will become better apparent hereinafter are achieved by a device for regenerating biological tissues and by the respective manufacturing method, characterized in that it comprises, particularly for the provision of the inner portion of the scaffold, the steps of:

injecting an aqueous suspension of collagen in a mold that defines a cavity having an elongated shape,

immersing said mold, containing said aqueous suspension of collagen, in a bath of liquid nitrogen along its longitudinal axis for freezing said aqueous suspension of collagen,

sublimating said aqueous suspension of collagen contained in said mold, for example in a freeze-dryer,

extracting from said mold the supporting element for regenerating biological tissues as obtained at the end of said sublimation step,

drying, for example in a dryer, said supporting element.

### BRIEF DESCRIPTION OF THE DRAWINGS

Further characteristics and advantages of the invention will become better apparent from the description of a preferred but not exclusive embodiment of a device for regenerating biological tissues and of the respective manufacturing method, according to the invention, illustrated by way of non-limiting example in the accompanying drawings, wherein:

FIG. 1 is a perspective view of a supporting element according to the invention;

FIG. 2 is a perspective view of the outer sheath of a regeneration device according to the invention;

FIG. 3 is a perspective view of a regeneration device according to the invention, constituted by an outer sheath and by two supporting elements;

FIG. 4 is a flowchart of the method of manufacturing the regeneration device according to the invention;

FIG. 5 is a flowchart of the step of manufacturing the supporting element according to the invention;

FIG. 6 is a perspective view of a mold used to provide the supporting element according to the invention;

FIG. 7 is a perspective view of a support for molds used to manufacture supporting elements according to the invention.

### WAYS OF CARRYING OUT THE INVENTION

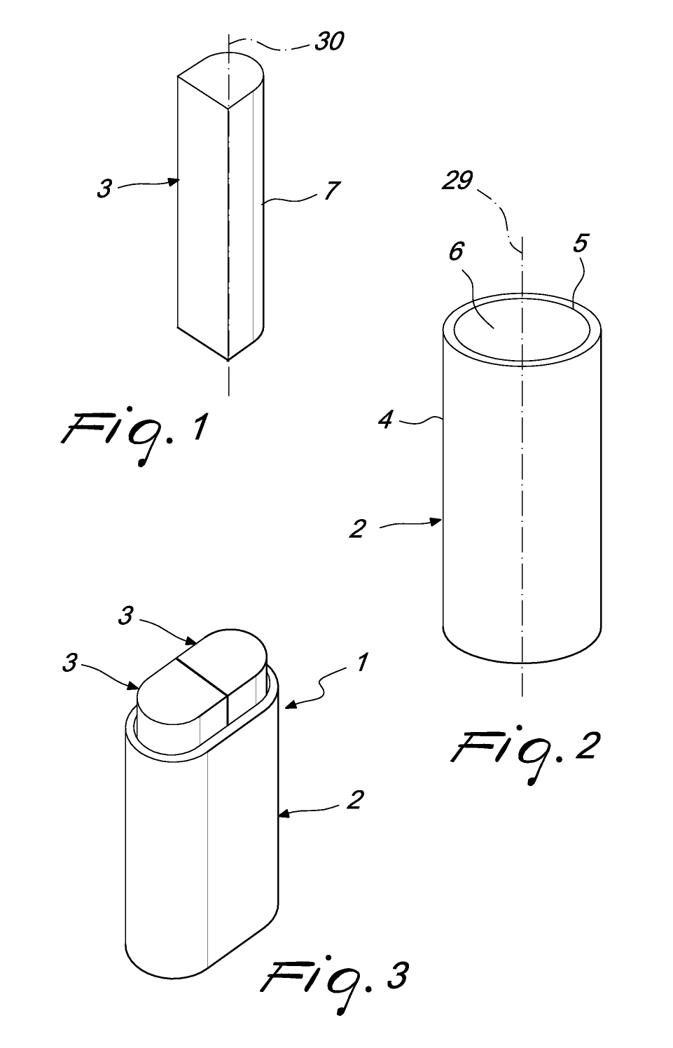

With reference to FIGS. 1 to 3, the regeneration device according to the invention, generally designated by the reference numeral 1, comprises an outer sheath 2 based on biocompatible material, preferably based on collagen, which can be interposed between the two endings of biological tissue to be regenerated.

More particularly, in a preferred configuration for applications aimed at regeneration of the spinal cord the outer sheath 2 has a substantially tubular shape.

The regeneration device 1 comprises at least one inner element 3 based on biocompatible material, preferably based on collagen, accommodated inside the outer sheath 2 to facilitate biological regrowth of the biological tissue.

Conveniently, for the application of regenerating the spinal cord, there are two inner elements 3 that lie substantially parallel to each other and along a preferred axis 30, which is parallel to the longitudinal axis 29 of the outer sheath 2, each one having a transverse cross-section shaped like a semielliptical lobe.

For containing two supporting elements 3 and reproducing the outer shape of the white matter of the spinal cord on which the regeneration device 1 is to be implanted, the outer sheath 2 has, once it has been implanted, a transverse cross-section with a substantially elliptical shape.

The two supporting elements 3 contained in it are thus arranged so as to simulate the inner structure of the spinal cord, constituted by two lobes, a right one and a left one, of gray matter.

Advantageously, the outer sheath 2 has a porous structure, so that its outer wall 4, having a higher relative density of collagen, has a reduced average pore size so as to form a region that is permeable to proteins and impermeable to cells.

Differently, the inner wall 5 of the outer sheath 2 can have a smaller volumetric fraction of solid and, therefore, a lower relative density of collagen, and a larger average pore size so as to constitute a region that is permeable to the cells that are present inside the cavity 6 of the outer sheath 2.

Conveniently, to allow a preferential cell migration from the cavity 6 of the outer sheath 2 toward the outer wall 4 through which inflow of cells from the outside is however blocked, the pores of the inner wall 5 can be oriented in a substantially radial direction with respect to the longitudinal axis 29.

As already mentioned, each supporting element 3 has an elongated shape along a preset direction with a transverse cross-section that is substantially semielliptical and simulates the shape of the gray matter of the corresponding lobe of the spinal cord.

As will be described in more detail hereinafter, the supporting element 3 has a relative density of collagen that is substantially constant in all directions.

Like the outer sheath 2, the supporting element 3 also has a porous structure with controlled porosity.

In this case, however, the pores of the supporting element 3 are oriented substantially longitudinally with respect to the longitudinal axis 29 of the outer sheath 2 to promote the regeneration of the biological tissue inside said pores where the regeneration device 1 is applied.

In particular, for applications related to the spinal cord, said longitudinal porosity is necessary in order to sustain the axial growth of the axons and of the Schwann cells' channels of the spinal cord.

In a particularly advantageous configuration, the outer sheath 2, directly after being manufactured and before its deformation necessary for its implantation, has an outside diameter that is preferably equal to 12 millimeters and an inside diameter that is preferably equal to 10 millimeters.

In the same advantageous configuration, the semielliptical lobes of the inner elements 3 have a major axis of the ellipse that is preferably equal to 12 millimeters and a minor axis of the ellipse that is preferably equal to 6 millimeters.

Moreover, the pore size of the supporting elements 3, for example comprised between 5 μm and 20 μm, is such as to promote axonal regrowth.

Advantageously, each one of the supporting elements 3, besides being provided with a porous structure based on collagen, can comprise additions of at least one among fibronectin, hyaluronic acid, elastin and fibrin.

With reference to the figures, the method 100 for manufacturing the regeneration device 1, according to the invention, comprises a step 101 of manufacturing the outer sheath 2 and a step 102 of manufacturing the inner elements 3.

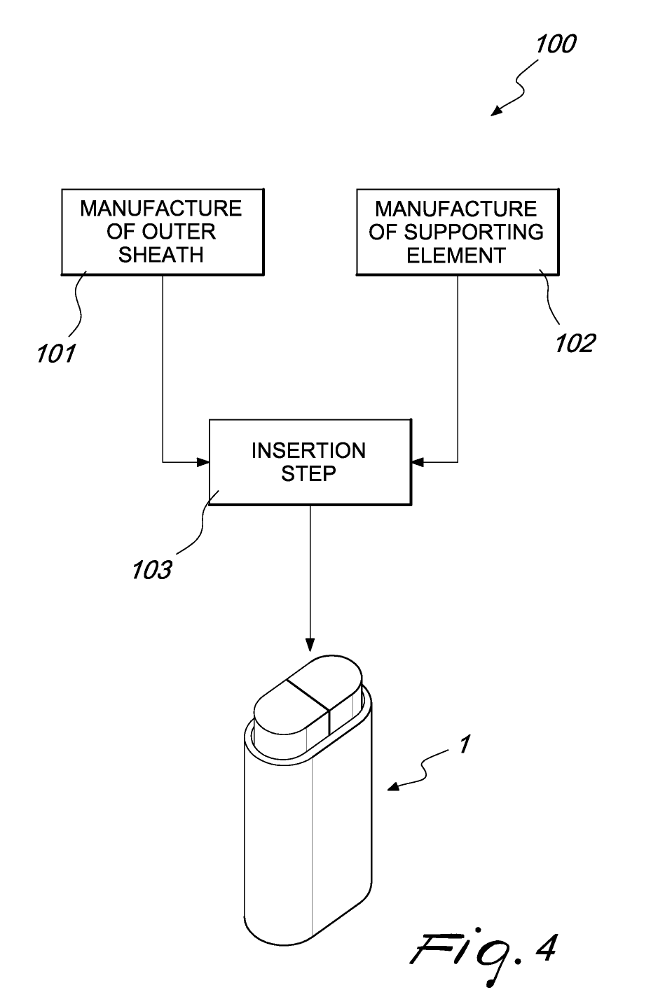

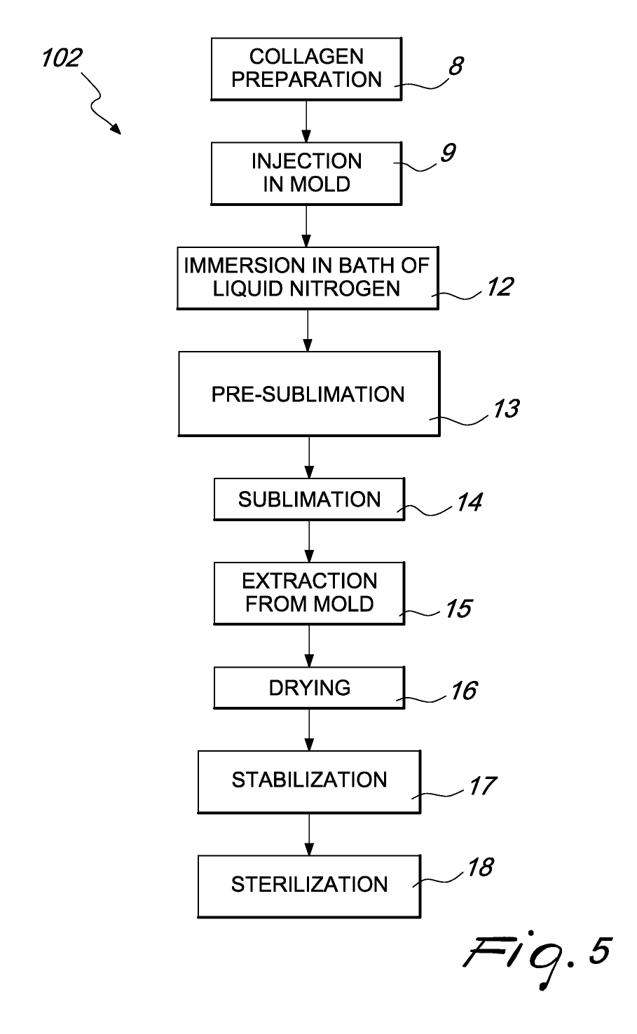

The manufacturing method 100, shown schematically in FIG. 4, comprises at least one step 103 of insertion of each supporting element 3 provided in the outer sheath 2.

More precisely, the manufacturing step 101 can be performed by means of a method known per se starting from an aqueous suspension of Type I fibrillar collagen, which is derived, for example, from cattle hide, and contains a high solid content, for example equal to 3% by weight.

This aqueous suspension is degassed by centrifugation, for example, for 12 minutes at 6000 rpm for eliminating the air introduced during mixing.

The suspension is then stored at a temperature of about 4° C. and, before use, is left for a few hours at ambient temperature, comprised between 18° C. and 20° C., so as to reduce its viscosity and thus facilitate the subsequent injection step.

In this way, the suspension is ready to be injected, for example by means of a graduated pipette, into a tubular mold made, for example, of PVC (polyvinyl chloride) or silicone.

Such tubular mold is subsequently sealed and inserted in a cylindrical support which comprises a cylindrical body screwed to a base with one end. The cylindrical support is made of copper or of a material with a similar heat conductivity and is necessary for vertical coupling to a rotor.

Such rotor subjects the tubular mold and the aqueous suspension of collagen contained therein to a rotation, about a specific axis of rotation, at a preset rate and for a preset time so as to cause a phenomenon of sedimentation of the collagen on the walls of the mold, thus producing the desired inner geometry of the structure of the outer sheath 2.

For obtaining an outer sheath 2 that runs along the longitudinal axis 29, the latter is made to coincide with the rotation axis of the mold. In particular, by adjustment of the rotation rate of the rotor the inside diameter of the outer sheath 2 is adjusted.

The fact that the collagen is in an aqueous suspension and, therefore, the fact of having components of sufficiently different density, allows complete removal of the collagen from the portion that surrounds the rotation axis of the mold, thus providing a hollow tubular structure.

More precisely, the tubular mold, which contains the aqueous suspension of collagen, is immersed, while still under rotation, in a bath of liquid nitrogen and frozen for a preset time, at the end of which the mold is extracted from said bath and rotation is stopped.

This freezing makes it possible to create ice crystals inside the sedimented collagen structure which are subsequently removed by sublimation and drying, thus providing the desired porous structure.

As regards the supporting element 3, it is obtained from the manufacturing step 103, which comprises the steps illustrated in the flowchart of FIG. 5.

More precisely, the manufacturing step 103 comprises a step 8 in which the preparation of the aqueous suspension of Type I fibrillar collagen occurs, which collagen is derived, for example, from cattle hide and contains a high solid content, for example equal to 3% by weight, from which suspension the desired supporting element 3 is to be obtained.

Subsequently, one moves on to step 9, in which this suspension is injected into a mold 11 made, for example, of PVC (polyvinyl chloride) or silicone.

With reference to FIG. 6, advantageously the mold 11 is constituted by a body 20 whose shape is substantially elongated along a predefined direction 21 and defines an inner cavity 22 having a transverse cross-section that is substantially shaped like a semielliptical lobe, which corresponds to the geometrical shape that the supporting element 3 shall have.

More precisely, said body 20 has a single opening 23, arranged at one of its ends, through which the injection of the aqueous suspension of collagen is performed.

The mold 11 further comprises a closing element 24, which can be coupled to the opening 23 to form a closed inner volume.

For allowing an easier grip of the mold 11, on the opposite side with respect to the opening 23 the base of the mold 11 has a protruding outer rim 25.

The mold 11 is then immersed over its whole length, which is preferably equal to 35 millimeters, in a container that contains liquid nitrogen at a very slow rate, for example, 0.1 mm/s, following a direction which is parallel to its main direction 21.

This immersion allows the development, along the longitudinal axis 30 of the supporting element 3, of a distinct thermal gradient associated with the transport of heat. Therefore the crystals, which are formed by solidification of the aqueous solution of collagen, have a shape which is elongated in the direction of heat transport, that coincides with the direction of immersion.

In order to treat simultaneously a plurality of molds and thus optimize the productivity of the manufacturing step 103, an adapted support 3 for a plurality of molds 11 is used.

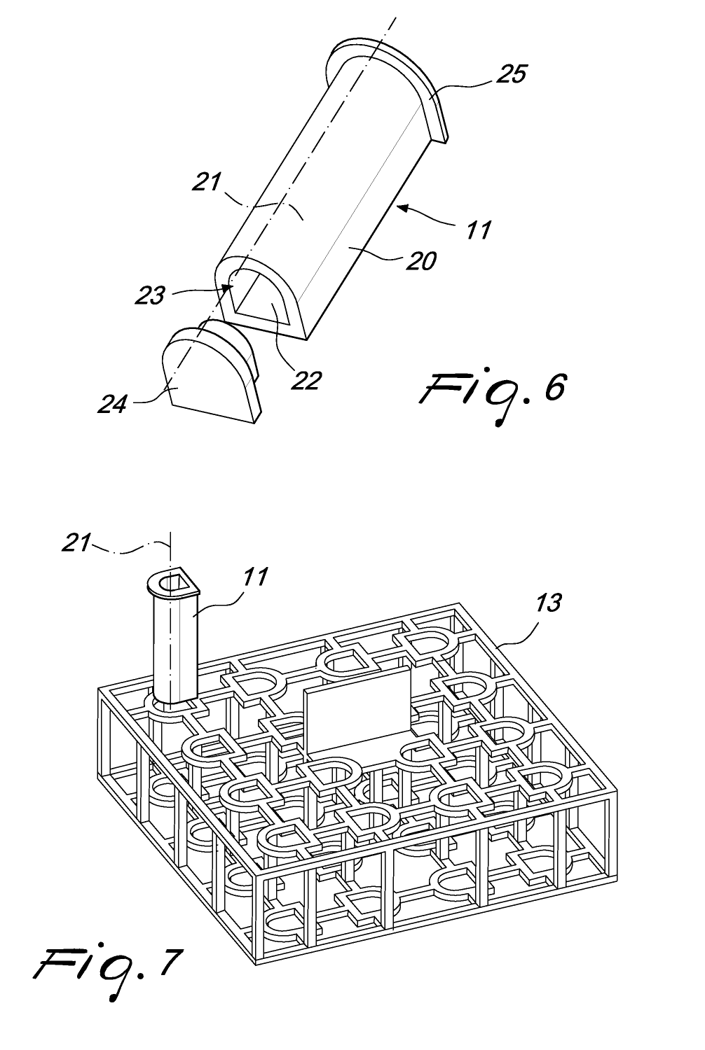

With reference to FIG. 7, the support 3, which allows the simultaneous immersion of a plurality of molds 11 in a container of liquid nitrogen, has a cage-like structure shaped substantially like a parallelepiped, on the upper face 26 of which a matrix of cavities 27 is formed adapted to accommodate the molds 11.

The cavities 27, which are also formed on the lower face 28 of the support 3, have such a geometric shape as to allow the insertion of the molds 11 and their retention in the correct position in order to avoid accidental movements on the plane at right angles to the direction of insertion.

The immersion step occurs preferably with rate control, so as to allow the desired development of a thermal gradient substantially along said preset direction and to form ice crystals inside the aqueous suspension of collagen. This speed control consists substantially in controlling the rate of immersion of the mold 11 in the bath of liquid nitrogen.

For this purpose, the rate control is aimed at maintaining an immersion rate that is substantially constant and preferably equal to 0.1 mm/s.

Once the mold 11 has been extracted from the bath of liquid nitrogen, after a time sufficient to achieve the freezing of the aqueous suspension of collagen along the whole length of the mold 11, said mold is introduced in a freeze-dryer, in which the pre-sublimation step 13 occurs.

In this step, the mold 11 is kept at a preset temperature, preferably equal to −40° C., for a preset time equal to 1 hour.

Subsequently, still inside the freeze-dryer, the sublimation step 14 occurs in which first the internal pressure of the freeze-dryer is lowered to a preset value, preferably equal to 200 mTorr, while the temperature is preferably kept equal to −40° C., and then, once said value of the pressure has been reached, the internal temperature of the freeze-dryer is raised to a preset value, preferably equal to 0° C.

The mold 11 is kept at such temperature for a preset time, preferably equal to 17 hours, and then the inside temperature of the freeze-dryer is raised to a preset value preferably equal to 20° C., for melting the previously obtained crystals.

Subsequently, once air has been injected into the freeze-dryer to restore atmospheric pressure inside it, the mold 11 is extracted from the freeze-dryer.

During the subsequent extraction step 15, the supporting element 3, thus obtained at the end of the sublimation step 14, is removed from said mold 11.

Finally, in step 16, the supporting element 3 is placed in a dryer to be dried.

Steps 14 and 16 are used to remove the aqueous component frozen during the immersion step 12, to obtain the desired porous structure of the supporting element 3.

Preferably, the supporting element 3 thus obtained can undergo a stabilization step 17 with the aim of reducing the degradation rate when implanted.

This stabilization step 17 occurs by means of a cross-linking treatment, which acts on the density of the cross-linking bonds that exist among the macromolecules of collagen.

More particularly, one of the procedures used can be DeHydroThermal Cross-Linking (DHT), which is a physical cross-linking treatment that does not provide for the use of cross-linking agents and, in particular, is performed in a vacuum oven for a period of time that varies from 24 to 48 hours at a temperature preferably equal to 121° C. with a pressure preferably equal to 100 mTorr. Other cross-linking procedures can include the use of carbodiimide, formaldehyde vapors, alone or together with DHT.

Finally, advantageously, the supporting element 3 undergoes a dry heat sterilization step 18, which makes it possible to avoid damaging and degrading the structural integrity of the supporting element 3. This dry heat sterilization treatment (Dry-Heat Sterilization, DHS) is preferably performed in a vacuum oven under standard conditions, i.e., for a period of time preferably equal to 2 hours and at a temperature preferably equal to 160° C.

The outer sheath 2, whose manufacture can comprise steps similar to the stabilization step 17 and sterilization step 18, and the supporting element 3 are provided by means of two independent processes and only when implantation occurs they are assembled together by insertion of at least one supporting element 3 in the outer sheath 2.

As already mentioned, the assembly and insertion operation 103 can entail a deformation of the outer sheath 2.

Since the structure of the spinal cord must be simulated, during the insertion step 103 two supporting elements 3 are inserted in the outer sheath 2 with the function of promoting the axonal regrowth of the right and left lobes of the lesioned spinal cord by providing the axons with an adequate physical and chemotactic support.

In practice it has been found that the device according to the invention fully achieves the intended aim, since the regeneration device makes it possible to facilitate biological regrowth of biological tissue.

In particular, in applications for regeneration of the spinal cord, the regeneration device, by having two supporting elements 3 that run substantially parallel to each other along the longitudinal axis of the outer sheath and each of which has a transverse cross-section shaped like a semielliptical lobe, is capable of simulating the inner architecture of the spinal cord constituted by two lobes, right and left, of gray matter.

Moreover, the fact that the outer sheath has, once implanted, a substantially oval transverse cross-section allows it to contain two supporting elements and, above all, to reproduce the outer shape of the white matter of the spinal cord on which the regeneration device is to be implanted.

Moreover, the fact that the outer wall of the outer sheath has a higher relative density of collagen and a reduced average pore size makes it a region that is permeable to proteins and impermeable to cells.

The fact is also not negligible that the inner wall of the outer sheath, by having a lower relative density of collagen and a larger average pore size, makes it possible to constitute a region that is permeable to the cells that are present inside the cavity of the outer sheath.

Moreover, the fact that the pores of the inner wall are oriented in a substantially radial direction with respect to the longitudinal axis of the outer sheath allows a preferential cell migration from the cavity of the outer sheath toward the outer wall, through which the entry of cells from the outside is however blocked.

Moreover, the fact that the pores of the supporting element are oriented substantially longitudinally with respect to the longitudinal axis of the outer sheath facilitates the regeneration of the biological tissue inside said pores where the regeneration device is applied and, particularly for applications related to the spinal cord, is designed to support the axial growth of the axons and of the Schwann cell channels of the spinal cord.

Moreover, the fact that the mold of the supporting element is constituted by a body having a substantially elongated shape along a preset direction and forming an inner cavity with a transverse cross-section that is shaped substantially like a semielliptical lobe defines the ideal geometric shape that the supporting element must have.

Further, control of the immersion rate allows the development, along the longitudinal axis of the supporting element, of a distinct thermal gradient associated with the transport of heat and causes the crystals that form by solidification of the aqueous suspension of collagen to have a shape that is elongated in the heat transport direction that coincides with the direction of immersion.

Moreover, the fact that the obtained supporting element undergoes a stabilization step makes it possible to reduce the degradation rate of the supporting element in vivo, increasing the density of the cross-linking bonds that exist among the macromolecules of collagen. This bioabsorption rate in vivo can be changed conveniently by varying the degree of cross-linking of the polymer that constitutes the scaffold (i.e., the collagen). These variations of the degree of cross-linking can be performed by varying the cross-linking technique (i.e., DHT, carbodiimide, formaldehyde) and/or the time and temperature of the stabilization process.

Also, the fact that the supporting element undergoes a dry heat sterilization step makes it possible to avoid damage and degradation of the chemical and physical qualities of the supporting element.

Finally, the fact that the outer sheath and the supporting element are provided with two independent processes makes it possible to have two structures with mutually different characteristics and properties, and since only when the implantation occurs they are assembled together by insertion of at least one supporting element in the outer sheath, makes it possible to obtain a regeneration device that can be used for regenerating tissues constituted by a plurality of parts with different requirements and characteristics of regrowth.

Although the device according to the invention has been conceived in particular to contain two supporting elements, it may be conceived to contain a single supporting element or more than two supporting elements, according to the requirements and the type of nerve on which the product shall be applied.

Moreover, although the supporting element has been conceived as having a substantially semielliptical transverse cross-section, it may nonetheless have transverse cross-sections of another shape.

Finally, although the device according to the invention has been conceived particularly for applications for regenerating the spinal cord, it may be used nonetheless, more generally, for regenerating peripheral nerves, tendons, bones, cartilages, vessels and so forth.

The regeneration device and the corresponding manufacturing method, thus conceived, are susceptible of numerous modifications and variations, all of which are within the scope of the appended claims; all the details may further be replaced with other technically equivalent elements.

In practice, the materials used, as well as the dimensions, may be any according to requirements and to the state of the art.

The disclosures in Italian Patent Application No. MI2009A001804 from which this application claims priority are incorporated herein by reference.

Where technical features mentioned in any claim are followed by reference signs, those reference signs have been included for the sole purpose of increasing the intelligibility of the claims and accordingly such reference signs do not have any limiting effect on the interpretation of each element identified by way of example by such reference signs.

1-18. (canceled)

19. A method for manufacturing a device for regenerating biological tissues, particularly for regenerating tissues of the central nervous system, comprising the steps of:
injecting an aqueous suspension of collagen in a mold that has a cavity with a substantially elongated shape along a predefined direction,
immersing said mold, containing said aqueous suspension of collagen, in a bath of liquid nitrogen along said preferred direction, for freezing said aqueous suspension of collagen,
sublimating said aqueous suspension of collagen contained in said mold,
extracting from said mold the supporting element for regenerating biological tissues as obtained at the end of said sublimation step,
drying said supporting element.

20. The manufacturing method according to claim 19, wherein said inner cavity has a transverse cross-section that is substantially shaped like a semielliptical lobe.

21. The manufacturing method according to claim 19, wherein said immersion step comprises a control of the rate of immersion of said mold in said liquid nitrogen bath so as to generate a thermal gradient substantially along said preset direction and form ice crystals within said aqueous suspension of collagen.

22. The manufacturing method according to claim 19, wherein during said immersion step said rate of immersion of said mold containing said aqueous suspension of collagen in said liquid nitrogen bath is preferably equal to 0.1 mm/s.

23. The manufacturing method according to claim 19, further comprising a step of pre-sublimation of said collagen suspension contained in said mold in a freeze-dryer at a preset temperature, preferably equal to −40° C., and for a preset time, preferably equal to 1 hour, said pre-sublimation step being performed between said immersion step and said sublimation step.

24. The manufacturing method according to claim 19, wherein said sublimation step comprises:
lowering the inside pressure of a freeze-dryer containing said aqueous solution to a preset value, preferably equal to 200 mTorr, while keeping said temperature preferably equal to −40° C.,
raising the inside temperature of said freeze-dryer to a preset value, preferably equal to 0° C.,
holding said inside temperature for a preset time, preferably equal to 17 hours,
raising the inside temperature of said freeze-dryer to a preset value, preferably equal to 20° C.,
injecting air into said freeze-dryer and restoring the atmospheric pressure inside said freeze-dryer.

25. The manufacturing method according to claim 19, further comprising a step for stabilizing said supporting element to reduce the degradation rate of said supporting element in vivo by means of a cross-linking treatment, said stabilization step being performed after said drying step.

26. The manufacturing method according to claim 19, further comprising a step of sterilization with dry heat of said supporting element.

27. The manufacturing method according to claim 19, further comprising a step for inserting at least one said supporting element in an outer sheath based on biocompatible material and having a substantially tubular shape, in order to obtain a device for regenerating biological tissues which can be interposed between the ends to be regenerated.

28. The manufacturing method according to claim 19, further comprising a step for inserting two supporting elements in an outer sheath based on biocompatible material and having a substantially tubular shape in order to obtain a device for regenerating biological tissues of the spinal cord, which can be interposed between the endings to be regenerated.

29. A supporting element for regenerating biological tissues, particularly for regenerating tissues of the central nervous system, which can be obtained from a manufacturing method according to claim 19.

30. The supporting element according to claim 29, wherein the supporting element has an elongated shape along a preferred axis with a substantially semielliptical transverse cross-section.

31. The supporting element according to claim 29, wherein the supporting element has a controlled structural porosity, with the pores oriented substantially longitudinally with respect to its preferred axis, so as to allow the biological tissue to grow inside said pores.

32. The supporting element according to claim 29, wherein the supporting element is based on collagen with additions of at least one of fibronectin, hyaluronic acid, elastin and fibrin.

33. A device for regenerating biological tissues, particularly for regenerating tissues of the central nervous system, obtainable by means of a manufacturing method according to claim 19.

34. The regeneration device according to claim 33, further comprising at least one supporting element for regenerating biological tissues, particularly for regenerating tissues of the central nervous system, the supporting element accommodated inside an outer sheath based on collagen and having a substantially tubular shape, the at least one supporting element being obtainable from a method for manufacturing a device for regenerating biological tissues, particularly for regenerating tissues of the central nervous system, comprising the steps of:
injecting an aqueous suspension of collagen in a mold that has a cavity with a substantially elongated shape along a predefined direction;
immersing said mold, containing said aqueous suspension of collagen, in a bath of liquid nitrogen along said preferred direction, for freezing said aqueous suspension of collagen;
sublimating said aqueous suspension of collagen contained in said mold; extracting from said mold the supporting element for regenerating biological tissues as obtained at the end of said sublimation step;
drying said supporting element.

35. The regeneration device according to claim 33, further comprising two supporting elements for regenerating biological tissues, particularly for regenerating tissues of the central nervous system, the supporting elements being accommodated within an outer sheath based on collagen and having a substantially tubular shape and being substantially mutually parallel along the longitudinal axis of said outer sheath, the supporting elements being obtainable from a method for manufacturing a device for regenerating biological tissues, particularly for regenerating tissues of the central nervous system, comprising the steps of:
injecting an aqueous suspension of collagen in a mold that has a cavity with a substantially elongated shape along a predefined direction;
immersing said mold, containing said aqueous suspension of collagen, in a bath of liquid nitrogen along said preferred direction, for freezing said aqueous suspension of collagen;
sublimating said aqueous suspension of collagen contained in said mold; extracting from said mold the supporting element for regenerating biological tissues as obtained at the end of said sublimation step;
drying said supporting element.

36. The regeneration device according to claim 34, wherein said outer sheath has a structural porosity in which the pores are oriented substantially radially with respect to its longitudinal axis so as to allow the growth of the biological tissue within said pores.
